(19) United States
(12) Patent Application Publication
(10) Pub. No.: US 2025/0316348 A1
(43) Pub. Date: Oct. 9, 2025

(54) USING MACHINE LEARNING FOR STANDARDIZING ELECTRONIC RECORDS

(71) Applicant: AccuCode Al, Inc., Little Rock, AR (US)

(72) Inventors: Nathan Myers, Little Rock, AR (US); Stephanie Howanietz, Little Rock, AR (US); Scott Roper, Maumelle, AR (US)

(21) Appl. No.: 18/628,456

(22) Filed: Apr. 5, 2024

(51) Int. Cl. G16H 10/60; G06F 16/11

(52) U.S. Cl. CPC G16H 10/60; G06F 16/116

(57) ABSTRACT

A method comprises receiving at least one data record associated with a patient, the data record including one or more data items represented as image data. Then, the method comprises pre-processing the at least one data record to enhance legibility of at least one data item of the one or more data items of the at least one data record. Then, the method comprises, using at least a first machine learning model, converting at least a portion of the pre-processed at least one data record into at least one machine-readable data record. Then, the method comprises identifying a standardized format. Then, the method comprises converting the machine-readable data record to the standardized record format and using at least a second machine learning model and assigning one or more predetermined activity codes to the at least one machine-readable data record in the standardized record format.

### BACKGROUND

Maintaining and securely sharing electronic records, such as electronic health records, often presents challenges. A lack of standardization often makes communication between systems difficult and records are often not uniformly digitized. For example, hospital systems must process handwritten records from health care professionals, such as doctors or nurses. These records may omit information, use idiosyncratic language specific to individual personnel, or may be difficult to read. While standardized formats for health records have been developed to mitigate these issues, they have not been universally adopted.

### SUMMARY

In some example embodiments, there may be provided a method including identifying a first machine learning model trained for conversion of data into a machine-readable format; identifying a second machine learning model trained for assigning one or more predetermined activity codes to input data records; receiving at least one data record associated with a patient, the data record including one or more data items represented as image data; pre-processing the at least one data record to enhance legibility of at least one data item of the one or more data items of the at least one data record; using at least the first machine learning model, converting at least a portion of the pre-processed at least one data record into at least one machine-readable data record; identifying a standardized format; converting the machine-readable data record to the standardized record format; using at least the second machine learning model, assigning one or more predetermined activity codes to the at least one machine-readable data record in the standardized record format.

In some variations, one or more of the features disclosed herein including the following features can optionally be included in any feasible combination. In some embodiments, the pre-processing comprises interpolating at least a portion of a text object into the data record. In some embodiments, the data record comprises a digital scan of a handwritten record, a scanned text document, or an electronic file. In some embodiments, the interpolating comprises using machine learning-implemented path tracing of the handwritten record to repair one or more characters of the handwritten record, increase legibility of one or more characters of the handwritten record, or insert one or more characters into the handwritten record. In some embodiments, the pre-processing comprises rotating the data record, rotating a text object of the data record, removing a visual artifact of the data record, adjusting a brightness, optical curve, or contrast of the data record, changing a bit depth of image data of the data record, or superimposing a visual aid onto image data of the data record. In some embodiments, rotating a text object of the data record is incorporated into a process for parallelizing a plurality of text objects of the data record. In some embodiments, the visual artifact is a scanned dust speck or scanned print error. In some embodiments, the visual aid is a bounding box. In some embodiments, converting at least the portion of the pre-processed data record comprises assigning a text object of the pre-processed data record to a field, wherein the field is based at least in part on an identifier of the bounding box. In some embodiments, the converting at least the portion of the pre-processed data record into a machine-readable format is performed using optical character recognition. In some embodiments, the standardized record format is Health Level 7 (HL7) Fast Healthcare Interoperability Resources (FHIR). In some embodiments, the method further comprises converting the machine-readable data record into a different version of HL7. In some embodiments, converting at least the portion of the pre-processed data record into a machine-readable format is implemented using an ensemble machine learning model. In some embodiments, converting at least the portion of the pre-processed data record comprises performing a spelling check or a grammar check. In some embodiments, the method further comprises generating an electronic report comprising an algorithmically-generated explanation of the assigning of the activity codes. In some embodiments, the method further comprises generating an electronic claim file from the machine-readable data record.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 illustrates a record processing environment, in accordance with some embodiments;

FIG. 2 illustrates a record processing system, in accordance with some embodiments;

FIG. 3 illustrates a record processing subsystem, in accordance with some embodiments;

FIG. 4 illustrates an activity code classification subsystem, in accordance with some embodiments;

FIG. 5 illustrates a process flow diagram, in accordance with some embodiments;

FIG. 6 illustrates a computer system; and

FIG. 7 is a diagram of an implementation of a transformer model.

### DETAILED DESCRIPTION

A record processing system uses machine learning techniques to process existing health records in various formats (e.g., scanned images of handwritten records) to generate standardized electronic medical records that may be adopted widely by electronic health record (EHR) systems. For example, generating the standardized electronic medical records includes algorithmically assigning billing codes to the records, which may benefit hospital systems as healthcare billing codes are often inaccurate. To do this, a first machine learning model processes an existing record to convert it into a machine-readable format. Next, a second machine learning model assigns one or more billing codes. Finally, the system converts the machine-readable file with assigned codes to a standardized EHR format.

In some examples, the first machine learning model is a large language model (LLM) which analyzes the lexical content of the medical record, then uses the analysis to generate a machine-readable record. Prior to machine learning analysis, the system may perform pre-processing tasks to improve readability or interpolate missing text. For example, the system may use handwriting analysis to fill in gaps in letters or trace letters. Other pre-processing tasks include sharpening or rotating at least a portion of the image.

The record processing system may then convert the machine-readable record into a standardized format, such as Health Level 7 (HL7). The standardized-format record may be back-converted into older HL7 versions to be compatible with legacy EHR systems.

In some examples, the second machine learning model analyzes the machine-readable record to assign one or more activity codes (e.g., medical billing codes) to it. The second machine learning model comprises, for example, an LLM or another type of transformer-based architecture. The second machine learning model also uses one or more additional machine learning classifiers, such as decision tree models or neural networks, to assist with assigning the activity codes.

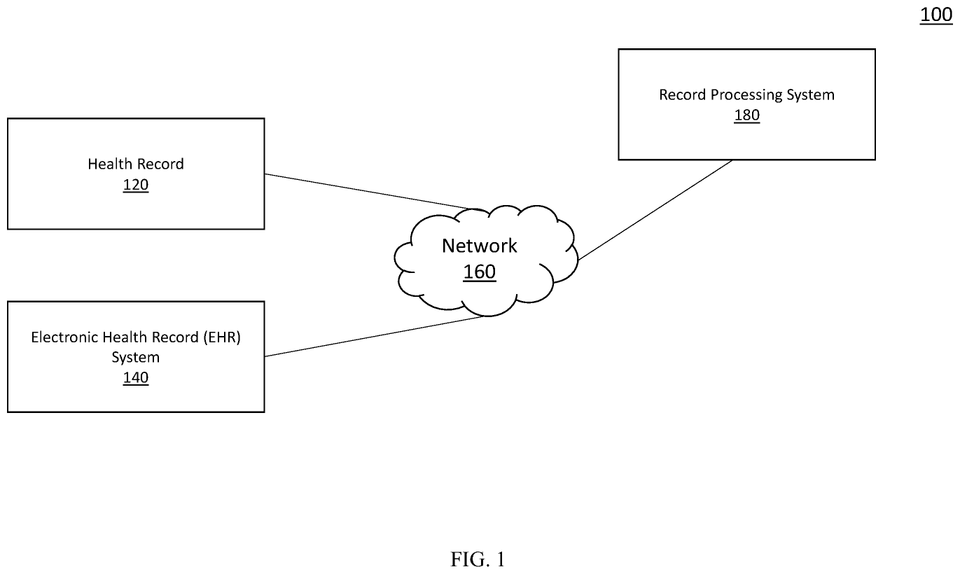

FIG. 1 illustrates a record processing environment 100, in accordance with some embodiments. The record processing environment 100 comprises a health record 120, an electronic health record (EHR) system 140, a network 160, and a record processing system 180.

The health record 120 includes medical and/or demographic information about a patient recorded by a health care professional. Medical information may include vital signs (e.g., blood pressure, heart rate), test results, medical history, allergy information, other health statistics, and/or medications taken or prescribed. Demographic information may include personal information, such as name, age, height, weight, race, sex, ethnicity, and/or residence. The health record 120 may also include insurance information, health facility information, and/or referrals by other health care practitioners.

The health record 120 comprises an electronic data record, such as an image or text file. For example, the health record 120 may be a digital scan of a handwritten record (e.g., from a scanner or photograph), a scanned text document, or an electronic file (e.g., a portable document format (PDF)). In another example, the health record 120 is a text transcription of audio (e.g., dictation) or video. The health record 120 may be produced by a healthcare professional (e.g., doctor, nurse, technician, medical assistant, physician assistant, or nurse practitioner).

The electronic health record (EHR) system 140 comprises a collection of patient and population electronically stored health information in a digital format. The electronic health record (EHR) system 140 may be shared across different health care settings. The data in the EHR system 140 may be shared through network-connected, enterprise-wide information systems.

Data accessible using the EHR system 140 may include demographic information, medical history, medication and allergies, immunization status, vital signs, personal statistics, and/or other data collected during medical procedures.

The network 160 allows the computing devices in the environment (e.g., EHR 140, record processing system 180), to electronically communicate with one another (e.g., accessing computing resources or sending messages). The network 160 may be a local area network (LAN), wide area network (WAN), or another type of network. The network 160 may be a wired or wireless network. The network 160 may enable computing devices of the record processing environment to communicate using a networking standard such as IEEE 802.3 (Ethernet) or 802.11 (wireless)

The record processing system 180 processes the health record into a standardized format usable by EHR system 140. The record processing system 180 uses a first machine learning model to determine the content (e.g., lexical content or meaning) of the health record 120 and produce a machine-readable version of the health record 120.

Before analysis with the first machine learning model, the record processing system 180 pre-processes health record 120 to generate inputs for the first machine learning model.

The record processing system 180 then, using the outputs from the first machine learning model, generates a machine-readable version of the record in a standardized format. The standardized format is a format enabling interpretation and sharing by EHR systems (e.g., EHR system 140). The standardized format may be, for example, a health level seven (HL7) format, such as HL7 Fast Healthcare Interoperability Resources (FHIR). The record processing system 180 can convert the standardized format record into an older format to interoperate with legacy EHR systems.

Once the standardized format record is generated, the record processing system 180 assigns one or more billing codes to the machine-readable version of the record using a second machine learning model.

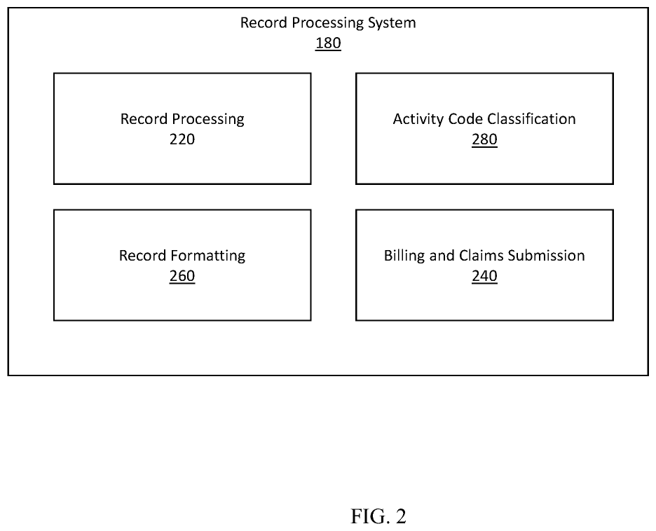

FIG. 2 illustrates the record processing system 180, in accordance with some embodiments. The record processing system 180 is configured to convert a health record (e.g., health record 120) into a standardized format ingestible by an EHR system (e.g., the EHR system 140).

The standardized format may be a global standard for transfer of clinical and health administration. The standardized format may include a syntax based on text elements. Text elements include delimiters (e.g., comma, semicolon, period, pipe, space character, newline character, carriage return, tilde character, tab character) and words or phrases representing categories of health information (e.g., patient name, insurance identifier, medical condition, time, date, visit information, patient identity, etc.) The standardized format may use or be based on a syntax such as extensible markup language (XML), JavaScript Object Notation (JSON), or Resource Description Framework (RDF). The standardized format may be a Health Level Seven (HL7) format, such as HL7 Fast Healthcare Interoperability Resources (FHIR). The standardized format may comprise an older or a newer version of HL7.

The record processing system 180 includes a record processing subsystem 220, a record formatting subsystem 260, an activity code classification subsystem 260, and a billing and claims submission subsystem 240. In some embodiments, record processing systems include additional or fewer modular components.

The record processing subsystem 220 pre-processes an input record to isolate text content of the input record. The record processing subsystem 220 uses machine learning or other image processing techniques to isolate the text.

The record formatting subsystem 260 may uses a first machine learning model to associate text in the pre-processed record with fields corresponding to a document in a standardized format (e.g., HL7). Then, the record formatting system 260 populates a standardized form with the fields and the associated text with the record. The record formatting subsystem 260 can down-convert or up-convert the record into an older or newer version of the standardized format, so that the standardized format record is interoperable with different EHR systems.

The activity code classification subsystem 280 assigns one or more activity codes to the standardized health record, using a second machine learning model. The second machine learning model is configured to analyze at least a portion of the text in one or more fields of the standardized format record to determine which activity codes to assign.

The billing and claims submission subsystem 240 uses an activity code to determine billing information and, in turn, generate and submit an insurance claim. The billing and claims submission sub-system 240 generates an electronic claim file in an appropriate format, such as X12 837. Then, the billing and claims submission subsystem 240 generates an invoice from the electronic claim file. The billing and claims submission subsystem 240 can convert the invoice into a format requested by a customer (e.g., paper or electronic). The billing and claims submission subsystem 240 submits an electronic claim form.

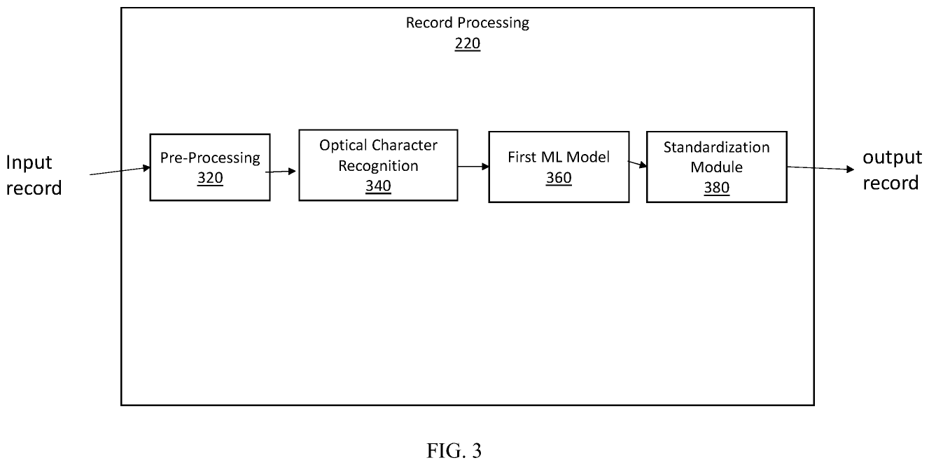

FIG. 3 illustrates the record processing subsystem 220, in accordance with an embodiment. The record processing subsystem 220 includes a pre-processing module 320, an optical character recognition module 340, a first machine learning model 360, and a standardization module 380. The record processing subsystem 220 processes an input record using these modules in series, to generate an output standardized format record.

The pre-processing module 320 performs one or more of the following pre-processing tasks to enhance legibility or readability of at least one data item of the health record (e.g., text or lettering) and/or isolate and/or digitize text objects in the health record. In some embodiments, the pre-processing module classifies one or more pages of a health record to determine which pages are relevant to demographic information, medical coding, or medical billing. Pages that cannot be classified into one of these three categories may be truncated or removed from the record.

The pre-processing module 320 identifies one or more relevant portions of the health record (e.g., the health record 120) from a scanned document or file package. For example, a user may upload a health record by capturing an image of a paper record sitting on a table. The pre-processing module 320 may isolate the portion of the image containing the paper record and remove the portion comprising the table. One or more image recognition techniques may be used to isolate the relevant portions. In some embodiments, a thresholding algorithm may be used to remove pixels corresponding to particular colors or shades. In some embodiments, a convolutional neural network (CNN) may be used to classify objects in the image to determine which objects to retain and which to remove. In some embodiments, edge detection techniques may be used.

The pre-processing module 320 detects letters or symbols in the health record. Techniques such as CNNs or computer vision algorithms identify handwritten or typed characters or symbols. The detected lexical content may be separated or isolated from non-lexical content of the health record.

In some embodiments, the pre-processing module 320 rotates or flips the document to allow the machine learning model to more easily process the text. For example, the pre-processing module 320 may be configured to rotate the document to align the text with a horizontal axis.

In some embodiments, the pre-processing module 320 rotates text of the health record, once the text has been identified or isolated. For example, the system may identify text and rotate it until it is aligned with (e.g., parallel to) a horizontal axis.

In some embodiments, the pre-processing module 320 uses a cleanup algorithm may locate print errors, dust specks, or other visual artifacts and remove them from the record. Cleanup may be performed using object recognition techniques, such as by identifying sharp contrasts in pixel values or edges in the record. Cleanup may also be performed using machine learning techniques (e.g., by processing the image with a CNN to identify artifacts.)

In some embodiments, the pre-processing module 320 may use a thresholding algorithm to alter the brightness, contrast, or geometric distortions of the image to make it more readable by the OCR. The pre-processing module 320 may modify a bit depth (e.g., a number of bits used to define an image) to facilitate this process. The thresholding algorithm may be binary (e.g., a pixel value may be selected as a threshold, and every value above may be black and every value may be white). Other thresholding algorithms may use histogram, clustering, entropy, object-attribute, or spatial methods, or comprise an Otsu algorithm.

In some embodiments, the pre-processing module 320 uses one or more handwriting analysis techniques to enhance lexical content in a handwritten medical record. For example, the pre-processing module 320 may determine a width of a handwritten stroke or use a ‘brush’ tool is used to trace over existing handwritten paths to fill any gaps introduced during the writing or scanning process.

In some embodiments, the pre-processing module 320 uses a computer vision technique to generate one or more bounding boxes to identify areas of the record to be processed via OCR or other object recognition techniques. The bounding boxes may identify areas of the record containing text or handwriting.

The optical character recognition module 340 processes the health record with an optical character recognition (OCR) model to generate digital text used to produce the standardized format record. The digital text may be generated from, for example, static text from a scanned image or converted from scanned handwriting.

For example, if the optical character recognition module 340 generates one or more bounding boxes, an OCR model may process the material inside the bounding box to convert the document into digital text.

In some embodiments, the bounding box is associated with an identifier (ID) which may categorize an object inside the health record (e.g., with respect to a field or type of information usually present in a health record). The OCR-generated digital text is assigned to a field based on the bounding box ID.

In some embodiments, the optical character recognition module 340 uses at least one of several techniques to perform OCR. For example, optical character recognition module 340 may use one or more machine learning algorithms (e.g., CNNs) to identify and digitize text in the health record.

In other embodiments, OCR uses matrix matching (e.g., comparing an image of a character or word in the document to an existing word or image)

In other embodiments, an OCR algorithm uses feature extraction to decompose characters into features, vectorize the features, and match the feature vectors with ground truth examples (e.g., stored in memory) to determine identities of characters, words, or symbols in the health record.

The first machine learning (ML) model 360 analyzes the lexical content of the pre-processed medical record to associate the digitized text of the record with fields of a standardized format health record. The fields may be associated with medical, personal, or demographic information of the patient, as well as information about medical personnel or one or more medical facilities associated with the patient.

Medical, personal, or demographic information about the patient includes age, height, weight, gender identity, sex, address, marital status, number of children, blood type, medical history, medications taken, substance use, and/or family medical history.

The first machine learning model 360 comprises a natural language processing and/or natural language understanding algorithm, such as a large learning model (LLM). In some embodiments, the first machine learning model 360 uses a transformer-based architecture. For example, the first machine learning model 360 may comprise a generative pre-trained transformer (GPT), or the like.

The first machine learning model 360 is trained using a large collection of medical records (e.g., handwritten or typed notes from health care providers), that have been pre-processed to comprise digitized text. The first machine learning model is trained to associate portions of the digitized text of the digitized records with particular categories or labels associated with fields of standardized record formats, such as those of HL7. In some embodiments, first machine learning model 360 is an ensemble model comprising multiple machine learning models, each capable of associating specific text from a health record with a subset (e.g., at least one) standardized format record field.

When the digitized text has been associated with fields, the standardization module 380 generates a standardized format record from the output of first machine learning model 360. In addition to populating the fields of the standardized format record with the associated digitized text, the standardization module 380 performs one or more verification or validation exercises to ensure the integrity and accuracy of the patient's standardized format record. For example, the standardization module 380 may format and provide the standardized format record to a third party to validate the patient's address, to make sure mail may be delivered to primary, secondary, and tertiary health insurance eligibility is verified to determine whether the patient has adequate insurance coverage. In some embodiments, the standardization module 380 retrieves information from public datasets to perform real-time updates and error correction of patient data. The standardization module may perform these actions periodically (e.g., every 30 days or 60 days).

In some embodiments, the standardization module 380 produces an output log of the demographics data, address verification, and insurance eligibility.

In some embodiments, the standardized format record is in an HL7 FHIR format. In some embodiments, the standardized record format is converted to HL7 v3, v2.5, v2.31, as required.

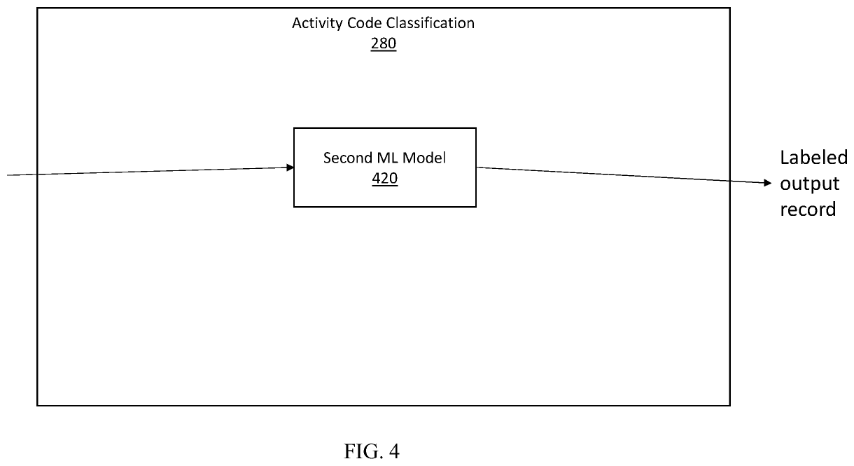

FIG. 4 illustrates the activity code classification subsystem 280, in accordance with some embodiments. In some embodiments, the activity code classification subsystem 280 processes the standardized format record with a second machine learning model 420 to assign the one or more activity codes.

The second machine learning model 420 comprises a natural language processing (NLP) model, such as a large language model (LLM), or another type of NLP model (e.g., with a transformer-based architecture). The second machine learning model 420 may be trained to associate standardized format records with particular records by associating at least a portion of text (e.g., relating to one or more fields of the standardized format record) with at least one activity code.

In some embodiments, when assigning the activity codes, the second machine activity code classification module 280 adheres to the standards established by the American Medical Association (AMA), the Centers for Medicare and Medicaid Services (CMS), or another health governing body (e.g., the American Academy of Professional Coders (AAPC), the American Health Information Management Association (AHIMA), and/or the American Society of Anesthesiologists (ASA).

In some embodiments, the activity code classification subsystem 280 generates a log of all the logic and sources used for assigning the activity codes. In some embodiments, the activity code classification subsystem assigns a charge code and value to one or more activity codes, depending on standards set by a payor insurance company.

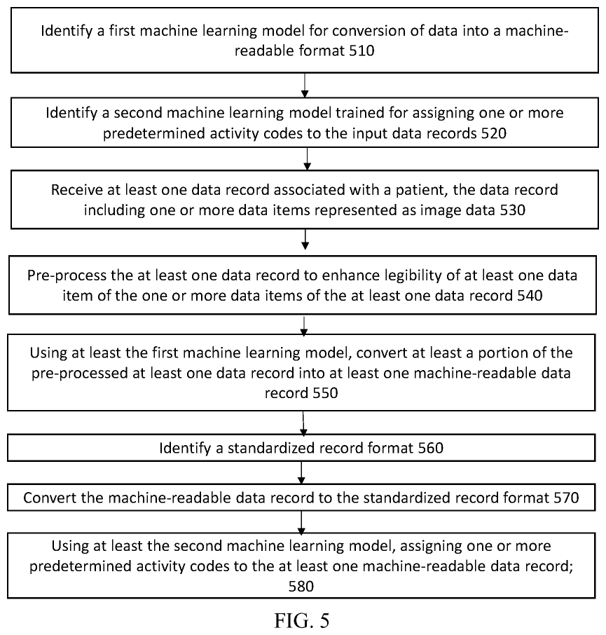

FIG. 5 illustrates a process 500 for generating a standardized format health record and assigning one or more activity codes to the standardized format record.

In a first operation 510, the system identifies a first machine learning model trained for conversion of data into a machine-readable format.

In a second operation 520, the system identifies a second machine learning model trained for assigning one or more predetermined activity codes to input data records.

In a third operation 530, the system receives at least one data record associated with a patient, the data record including one or more data items represented as image data. The data record is a health data record. The data record includes demographic data or personal data about the patient.

In a fourth operation 540, the system pre-processes the at least one data record to enhance legibility of at least one data item of the one or more data items of the at least one data record. The pre-processing comprises interpolating at least a portion of a text object into the data record. The interpolating comprises using machine learning-implemented path tracing of the handwritten record to repair one or more characters of the handwritten record, increase legibility of one or more characters of the handwritten record, or insert one or more characters into the handwritten record. The pre-processing comprises rotating the data record, rotating a text object of the data record, removing a visual artifact of the data record, adjusting a brightness, optical curve, or contrast of the data record, changing a bit depth of image data of the data record, or superimposing a visual aid onto image data of the data record. Rotating a text object of the data record may be incorporated into a process for parallelizing a plurality of text objects of the data record. The visual artifact may be a scanned dust speck or scanned print error. The visual aid may be a bounding box. Converting at least the portion of the pre-processed data record comprises assigning a text object of the pre-processed data record to a field, wherein the field is based at least in part on an identifier of the bounding box.

In a fifth operation 550, using at least the first machine learning model, the system converts at least a portion of the pre-processed at least one data record into at least one machine-readable data record (e.g., by digitizing the text of the data record); The converting at least the portion of the pre-processed data record into a machine-readable format is performed using optical character recognition. In some embodiments, converting at least the portion of the pre-processed data record into a machine-readable format is implemented using an ensemble machine learning model. Converting at least the portion of the pre-processed data record into a machine-readable format comprises performing a spelling check or a grammar check.

In a sixth operation 560, the system identifies a standardized record format. The standardized format may be Health Level 7 (HL7) Fast Healthcare Interoperability Resources (FHIR).

In a seventh operation 570, the system converts the machine-readable data record to the standardized record format. In some embodiments, the system converts the machine-readable data record into a different version of HL7.

In an eighth operation 580, using at least the second machine learning model, the system assigns one or more predetermined activity codes to the at least one machine-readable data record. Assigning the one or more predetermined activity codes comprises generating an electronic report with an algorithmically-generated explanation of the assigning of the activity codes. In some embodiments, the system generates an electronic claim file from the machine-readable data record.

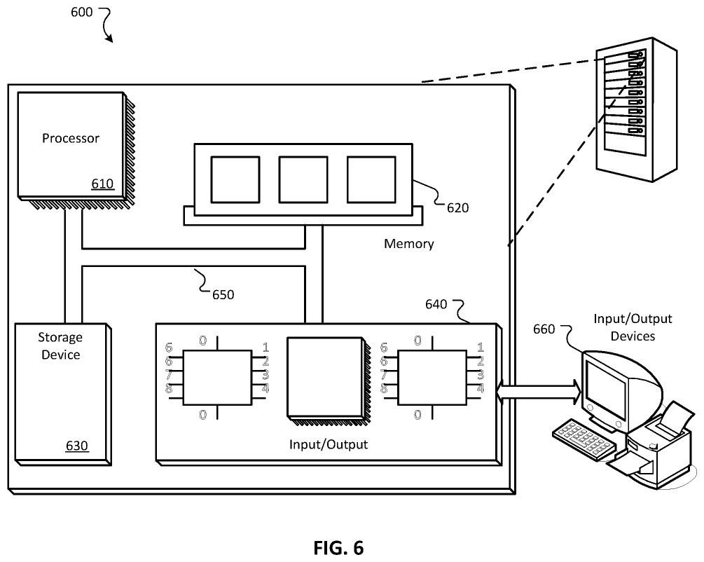

FIG. 6 is a block diagram of an example computer system 600. For example, FIG. 1 could be an example of the system 600 described here, as could a computer system used by any of the users who access resources of FIGS. 1-4 as shown in FIG. 1. The system 600 includes a processor 610, a memory 620, a storage device 630, and one or more input/output interface devices 640. Each of the components 610, 620, 630, and 640 can be interconnected, for example, using a system bus 650.

The processor 610 is capable of processing instructions for execution within the system 600. The term “execution” as used here refers to a technique in which program code causes a processor to carry out one or more processor instructions. In some implementations, the processor 610 is a single-threaded processor. In some implementations, the processor 610 is a multi-threaded processor. In some implementations, the processor 610 is a quantum computer. The processor 610 is capable of processing instructions stored in the memory 620 or on the storage device 630. The processor 610 may execute operations such as [general topic(s) of application].

The memory 620 stores information within the system 600. In some implementations, the memory 620 is a computer-readable medium. In some implementations, the memory 620 is a volatile memory unit. In some implementations, the memory 620 is a non-volatile memory unit.

The storage device 630 is capable of providing mass storage for the system 600. In some implementations, the storage device 630 is a non-transitory computer-readable medium. In various different implementations, the storage device 630 can include, for example, a hard disk device, an optical disk device, a solid-state drive, a flash drive, magnetic tape, or some other large capacity storage device. In some implementations, the storage device 630 may be a cloud storage device, e.g., a logical storage device including one or more physical storage devices distributed on a network and accessed using a network, such as the network shown in FIG. 1. The input/output interface devices 640 provide input/output operations for the system 600. In some implementations, the input/output interface devices 640 can include one or more of a network interface devices, e.g., an Ethernet interface, a serial communication device, e.g., an RS-232 interface, and/or a wireless interface device, e.g., an 802.11 interface, a 3G wireless modem, a 4G wireless modem, etc. A network interface device allows the system 600 to communicate, for example, transmit and receive data such as electronic health record data as shown in FIG. 1, e.g., using the network shown in FIG. 1. In some implementations, the input/output device can include driver devices configured to receive input data and send output data to other input/output devices, e.g., keyboard, printer and display devices 660. In some implementations, mobile computing devices, mobile communication devices, and other devices can be used.

Referring to FIG. 1, the record processing environment components can be realized by instructions that upon execution cause one or more processing devices to carry out the processes and functions described above, for example, processing and standardizing electronic health records. Such instructions can include, for example, interpreted instructions such as script instructions, or executable code, or other instructions stored in a computer readable medium.

A record processing system as shown in FIG. 1 can be distributively implemented over a network, such as a server farm, or a set of widely distributed servers or can be implemented in a single virtual device that includes multiple distributed devices that operate in coordination with one another. For example, one of the devices can control the other devices, or the devices may operate under a set of coordinated rules or protocols, or the devices may be coordinated in another fashion. The coordinated operation of the multiple distributed devices presents the appearance of operating as a single device.

In some examples, the system 600 is contained within a single integrated circuit package. A system 600 of this kind, in which both a processor 610 and one or more other components are contained within a single integrated circuit package and/or fabricated as a single integrated circuit, is sometimes called a microcontroller. In some implementations, the integrated circuit package includes pins that correspond to input/output ports, e.g., that can be used to communicate signals to and from one or more of the input/output interface devices 640.

Although an example processing system has been described in FIG. 6, implementations of the subject matter and the functional operations described above can be implemented in other types of digital electronic circuitry, or in computer software, firmware, or hardware, including the structures disclosed in this specification and their structural equivalents, or in combinations of one or more of them. Implementations of the subject matter described in this specification, such as storing, maintaining, and displaying artifacts can be implemented as one or more computer program products, i.e., one or more modules of computer program instructions encoded on a tangible program carrier, for example a computer-readable medium, for execution by, or to control the operation of, a processing system. The computer readable medium can be a machine readable storage device, a machine readable storage substrate, a memory device, or a combination of one or more of them.

The term “system” may encompass all apparatus, devices, and machines for processing data, including by way of example a programmable processor, a computer, or multiple processors or computers. A processing system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Computer readable media suitable for storing computer program instructions and data include all forms of non-volatile or volatile memory, media and memory devices, including by way of example semiconductor memory devices, e.g., EPROM, EEPROM, and flash memory devices; magnetic disks, e.g., internal hard disks or removable disks or magnetic tapes; magneto optical disks; and CD-ROM, DVD-ROM, and Blu-Ray disks. The processor and the memory can be supplemented by, or incorporated in, special purpose logic circuitry. Sometimes a server (e.g., record processing system as shown in FIG. 6) is a general purpose computer, and sometimes it is a custom-tailored special purpose electronic device, and sometimes it is a combination of these things. Implementations can include a back end component, e.g., a data server, or a middleware component, e.g., an application server, or a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network such as the network shown in FIG. 1. Examples of communication networks include a local area network (“LAN”) and a wide area network (“WAN”), e.g., the Internet.

Machine learning models herein may use one or more natural language processing and/or natural language understanding algorithms.

In some embodiments, the machine learning models may be large language models. A large language model (LLM) is a language model may learn statistical relationships from text documents to be able to generate, interpolate, or predict language (e.g., in text form). may be built with a transformer-based architecture. In many cases, LLMs are built using decoder-only architectures.

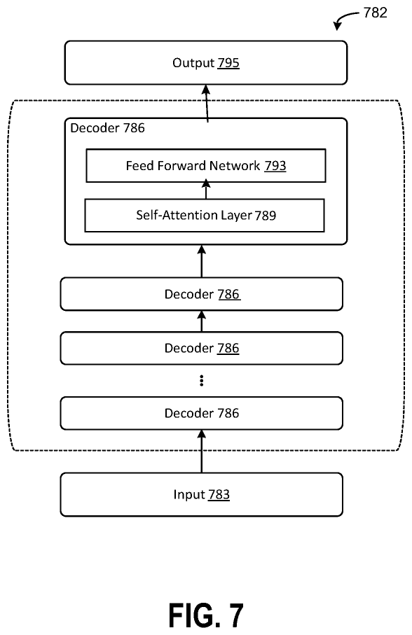

Referring now to FIG. 7, illustrated is a diagram of an implementation of a machine learning model. More specifically, illustrated is a diagram of an implementation of a decoder-only transformer model 782. In some example embodiments, the decoder-only transformer model 782 may implement the first machine learning model 360 and/or the second machine learning model. As will be described in more detail, the transformer model 782 may include a self-attention mechanism to capture the relative significance and relationship between different portions of an input 783. For instance, in cases where the input 783 is an image (e.g., of the health record 120), the self-attention mechanism of the transformer model 782 may capture the relative significance and relationship amongst different portions (or patches) of the image when generating an output 795 that includes, for example, one or more labels classifying one or more objects present in the image. While the transformer model 782 includes certain features as described herein, these features are provided for the purpose of illustration and are not intended to limit the present disclosure.

As shown in FIG. 7, the transformer model 782 may include a decoder stack having a plurality of decoders 786 (or decoding layers). In the example shown in FIG. 7, the input 783 (e.g., the embedding of each individual portion of the input 783) flows through every decoder 486 in the decoder stack.

Referring again to FIG. 7, the decoder stack may decode the input 783 to generate the output 795, with each decoder 786 in the decoder stack successively decoding the output of the previous decoder 786. For example, the first decoder 786 in the decoder stack may generate a first decoding of the input 783 (e.g., the embedding of each individual portion of the input 783) while the next decoder 786 in the decoder stack may generate a second decoding of the first decoding. As shown in FIG. 7, each decoder 786 may include a self-attention layer 789 and a feed forward network 793. The self-attention layer 789 of the decoder 786 may enable the decoder 786 to generate a context-aware decoding of the input 783 where the decoding for each individual portion of the input 783 incorporates weighted values corresponding to one or more preceding portions of the input 783. For example, in cases where the input 483 is an image, the self-attention layer 485 may determine the relationship between different portions of the image. In some cases, the self-attention layer 789 may include a multi-headed attention mechanism, with each head applying a different set of weights (e.g., query, key, and value weight matrices) for incorporating the other portions of the input 783. It should be appreciated that the weights (e.g., query, key, and value weight matrices) applied by the self-attention layer 789 may be learned during the training of the transformer model 782.

Transformers convert input text into tokens, which are processed by an encoder and/or decoder. Using a modification of byte-pair encoding, in the first step, all unique characters (including blanks and punctuation marks) are treated as an initial set of n-grams (i.e. initial set of uni-grams). Successively the most frequent pair of adjacent characters is merged into a bi-gram and all instances of the pair are replaced by it. All occurrences of adjacent pairs of (previously merged) n-grams that most frequently occur together are then again merged into even lengthier n-gram repeatedly until a vocabulary of prescribed size is obtained. Token vocabulary consists of integers, spanning from zero up to the size of the token vocabulary. New words can always be interpreted as combinations of the tokens and the initial-set uni-grams.

A token vocabulary based on the frequencies extracted from mainly English corpora uses as few tokens as possible for an average English word.

To find which tokens are relevant to each other within the scope of the context window, the attention mechanism calculates “soft” weights for each token, more precisely for its embedding, by using multiple attention heads, each with its own “relevance” for calculating its own soft weights.

A model may, for example, be pre-trained to predict how the segment continues (autoregressive), or what is missing in the segment, given a segment from its training dataset. An autoregressive model, for example, given a segment “I like to eat”, may predict “ice cream”, or “sushi.” A model trained to predict what is missing, for example, given a segment “I like to [______] [______] cream”, may predict that “eat” and “ice” are missing.

Models may be trained on auxiliary tasks which test their understanding of the data distribution, such as Next Sentence Prediction (NSP), in which pairs of sentences are presented and the model must predict whether they appear consecutively in the training corpus. During training, regularization loss is also used to stabilize training. However regularization loss is usually not used during testing and evaluation.

The transformer building blocks are scaled dot-product attention units. For each attention unit, the transformer model learns three weight matrices: the query weights, the key weights, and the value weights. For each token, the input token representation is multiplied with each of the three weight matrices to produce a query vector, a key vector, and a value vector. Attention weights may be calculated using the query and key vectors: the attention weight between two tokens is the dot product between the query and key elements respectively corresponding to each token. The attention weights may be divided by the square root of the dimension of the key vectors, which stabilizes gradients during training, and passed through a softmax which normalizes the weights. The fact that the query weights and key weights are different matrices allows attention to be non-symmetric. The output of the attention unit for a token is the weighted sum of the value vectors of all tokens, weighted by the attention from the token to each other token.

One set of query, key, and value matrices may comprise an attention head. Each layer in a transformer model may have multiple attention heads. Each attention head generates weights signifying attention tokens that are relevant in some way to each token. Using multiple attention heads allow the model to do this for different definitions of “relevance.” Each of many transformer attention heads may each encode different relevance relations that are meaningful to humans. For example, some attention heads can attend mostly to a next word in a sequence, while others mainly attend from verbs to their direct objects. The computations for each attention head can be performed in parallel, which allows for fast processing. The outputs for the attention layer are concatenated to pass into the feed-forward neural network layers.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

1. A method, comprising:
identifying a first transformer-based machine learning model trained for conversion of data into a machine-readable format;
identifying a second transformer-based machine learning model trained for assigning one or more predetermined activity codes to input data records;
receiving at least one data record associated with a patient, the data record including one or more data items represented as image data, the image data comprising a digital scan of a handwritten record;
pre-processing the at least one data record to enhance legibility of at least one data item of the one or more data items of the at least one data record, wherein the pre-processing comprises interpolating at least a portion of a text object into the data record, wherein the interpolating comprises using machine learning-implemented path tracing of the handwritten record to repair one or more characters of the handwritten record;
using at least the first transformer-based machine learning model, converting at least a portion of the pre-processed at least one data record into at least one machine-readable data record, the converting at least the portion of the pre-processed at least one data record into at least one machine-readable data record comprising:
processing the pre-processed at least one data record using a decoder layer of the first transformer-based machine learning model, the decoder layer comprising a self-attention layer, wherein the self-attention layer enables the decoder to generate a context-aware decoding of the pre-processed at least one data record;
identifying a standardized format;
converting the machine-readable data record to the standardized record format;
using at least the second machine learning model, assigning one or more predetermined activity codes to the at least one machine-readable data record in the standardized record format.

2-3. (canceled)

4. The method of claim 1, wherein the interpolating further comprises using machine learning-implemented path tracing of the handwritten record to increase legibility of one or more characters of the handwritten record, or to insert one or more characters into the handwritten record.

5. The method of claim 1, wherein the pre-processing comprises at least one of: rotating the data record, rotating a text object of the data record, removing a visual artifact of the data record, adjusting a brightness, optical curve, or contrast of the data record, changing a bit depth of image data of the data record, or superimposing a visual aid onto image data of the data record.

6. The method of claim 5, wherein rotating a text object of the data record is incorporated into a process for parallelizing a plurality of text objects of the data record.

7. The method of claim 5, wherein the visual artifact is a scanned dust speck or scanned print error.

8. The method of claim 5, wherein the visual aid is a bounding box.

9. The method of claim 8, wherein converting at least the portion of the pre-processed data record comprises assigning a text object of the pre-processed data record to a field, wherein the field is based at least in part on an identifier of the bounding box.

10. The method of claim 1, wherein the converting at least the portion of the pre-processed data record into a machine-readable format is performed using optical character recognition.

11. The method of claim 1, wherein the standardized record format is Health Level 7 (HL7) Fast Healthcare Interoperability Resources (FHIR).

12. The method of claim 11, further comprising converting the machine-readable data record into a different version of HL7.

13. The method of claim 1, wherein converting at least the portion of the pre-processed data record into a machine-readable format is implemented using an ensemble machine learning model.

14. The method of claim 1, wherein converting at least the portion of the pre-processed data record comprises performing a spelling check or a grammar check.

15. The method of claim 1, further comprising generating an electronic report comprising an algorithmically-generated explanation of the assigning of the activity codes.

16. The method of claim 1, further comprising generating an electronic claim file from the machine-readable data record.

17. A computer-implemented method of training a transformer-based machine learning model, comprising:
obtaining data representing a set of digital health records;
applying one or more transformations to one or more of the digital health records to create a pre-processed set of digital health records, the transformations comprising:
applying optical character recognition to at least one digital health record,
classifying one or more portions of the at least one digital health record using a thresholding algorithm, and
rotating or interpolating one or more characters identified in the at least one digital health record, wherein the interpolating comprises using machine learning-implemented path tracing of a handwritten record of the digital health record to repair one or more characters of the handwritten record;
creating a training set comprising the pre-processed set of digital health records; and
training the transformer-based machine learning model, using the training set, to convert at least the portion of the pre-processed at least one data record into at least one machine-readable data record, the converting at least the portion of the pre-processed at least one data record into at least one machine-readable data record comprising:
processing the pre-processed at least one data record using a decoder layer of the first transformer-based machine learning model, the decoder layer comprising a self-attention layer, wherein the self-attention layer enables the decoder to generate a context-aware decoding of the pre-processed at least one data record.

18. The method of claim 1, wherein the first transformer-based machine learning model is trained to associate portions of digitized text of a digitized record with particular categories or labels associated with one or more fields of the standardized format.

19. The method of claim 1, wherein assigning one or more predetermined activity codes to the at least one machine-readable data record in the standardized record format is performed at least in part by associating at least a portion of text relating to one or more fields of the standardized format record with at least one activity code of the one or more predetermined activity codes.

20. The method of claim 18, wherein the associating the portions of the digitized text of the digitized record with the particular categories or labels is performed at least in part by using the self-attention layer to capture a relative significance and relationship amongst different portions or patches of the image data.
